(19) United States
(12) Patent Application Publication
(10) Pub. No.: US 2022/0401253 A1
(43) Pub. Date: Dec. 22, 2022

(54) DEVICE FOR PREVENTING SLEEP APNEA AND SNORING

(71) Applicant: Diego Gonzalez Joven, La Jolla, CA (US)

(72) Inventor: Diego Gonzalez Joven, La Jolla, CA (US)

(21) Appl. No.: 17/351,688

(22) Filed: Jun. 18, 2021

(51) Int. Cl. A61F 5/56; A61N 1/05

(52) U.S. Cl. CPC A61F 5/56; A61N 1/0546

(57) ABSTRACT

A device for preventing sleep apnea and snoring, which is shaped like a semi-rigid whip. This whip has a curve that goes according to the anatomy of the base of the nasal cavity. At the wavy end of the whip is a design in the shape of the letter M that keeps the device fixed to the nostrils and, in turn, helps expand wing and triangular cartilages.

### FIELD OF INVENTION

This invention refers to a device for preventing sleep apnea and snoring used in the medical and health fields.

### SUMMARY OF INVENTION

An aspect of the invention involves a device for preventing sleep apnea and snoring is used to improve sleep and prevent all the problems that sleep apnea and snoring cause.

Another aspect of the invention involves a device for preventing sleep apnea and snoring, which is shaped like a semi-rigid whip of approximately 10 centimeters. This whip has a curve that goes according to the anatomy of the base of the nasal cavity. At the wavy end of the whip is a design in the shape of the letter M that keeps the device fixed to the nostrils and, in turn, helps expand wing and triangular cartilages.

### BACKGROUND OF THE INVENTION

The main cause of snoring and sleep apnea is because the soft palate relaxes and makes contact with the throat wall obstructing the free passage of oxygen. The main function of the device of the present invention is that by putting a splint in the soft palate with one end of the flexible longitudinal portion of the device, the device allows oxygen flow to the lungs to be free.

There are currently devices such as the one described in patent DE10028013 published on Dec. 13, 2001, however, such a device, due to its design and thickness, does not allow its shape to be retained and therefore, the function of eliminating muscle recession and damping vibrations does not allow for retaining its shape and therefore the function of eliminating muscle recession, and damping vibrations are not possible.

While the device of this invention solves such problems because the cylindrical shape makes it possible to have greater rigidity. Also, the flexible internal reinforcement allows the function of the device to be more effective. The design of our device goes more by the internal anatomy of the nasal cavity. Our design by pressing the muscle makes it more comfortable.

The advantages of the invention will become evident from the detailed description of the preferred modalities of the invention and accompanying drawings.

### BRIEF DESCRIPTION OF THE DRAWINGS

FIG. 1 illustrates a detail of the modular terminals of the device of this invention.

FIG. 2 illustrates a perspective view of the device of the present invention.

FIG. 3 illustrates a perspective view of the front body of the device of the present invention.

FIG. 4 illustrates a front view of the front body of the device of this invention.

FIG. 5 illustrates the inside of the front body of the device of this invention.

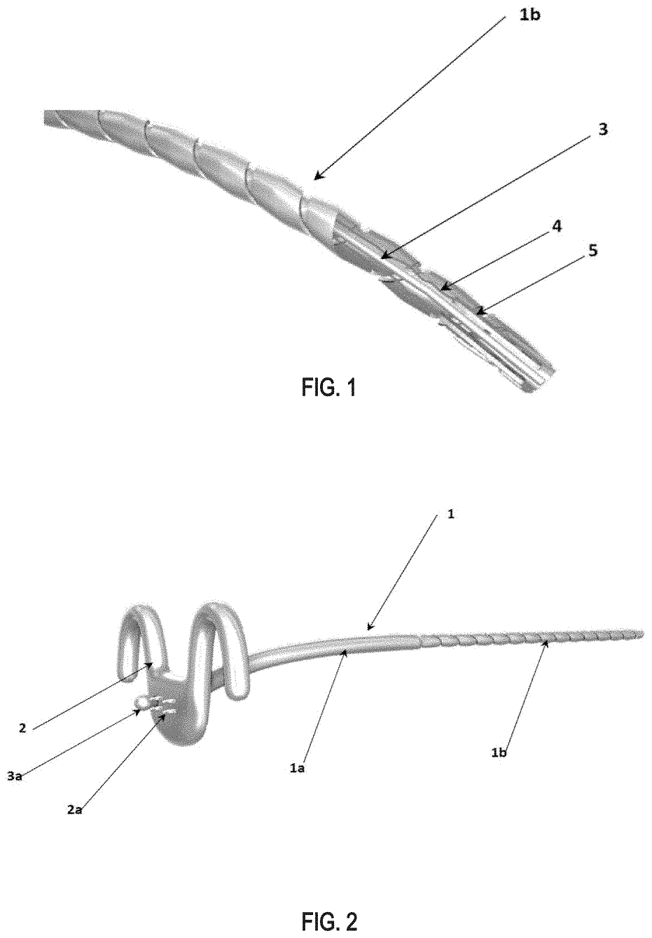

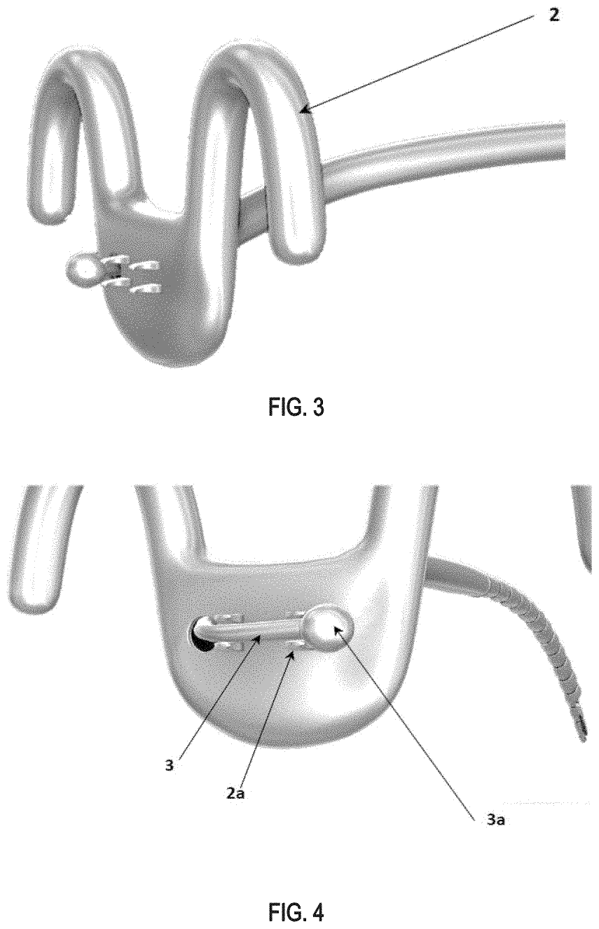

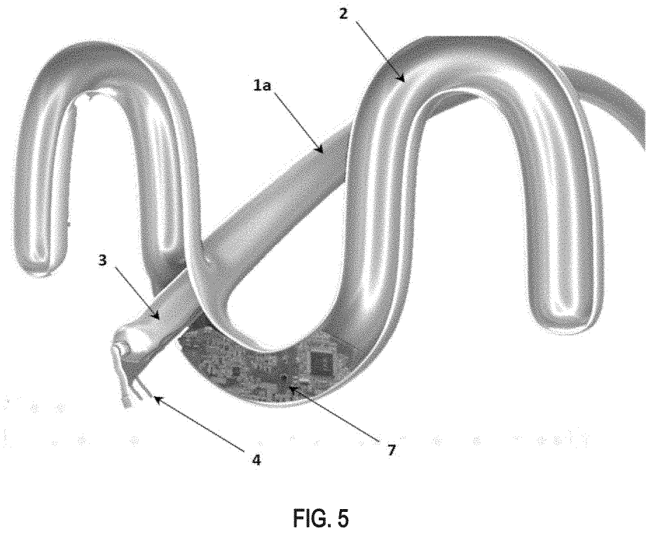

### DETAILED DESCRIPTION OF THE INVENTION

The device for the prevention of sleep apnea and snoring is in the form of the main body elongated within a range of 5 cm to 10 cm. This elongated main body can generate a curve consistent with the anatomy of the base of the nasal cavity. At the first end of the elongated main body is a design in the form of the letter “M” that keeps the device fixed to the nostrils and, in turn, helps to expand the wing and triangular cartilages.

More specifically, according to the figures illustrated, the device for preventing sleep apnea and snoring, consists of an elongated main body 1 with a rigid longitudinal portion 1a and a flexible longitudinal portion 1b that is removed from the rigid longitudinal portion 1a, having said elongated main body 1 a tension channel 4 extending over the entire length of it.

The elongated main body also comprises a front body 2 arranged at one end of the rigid longitudinal portion 1a of said elongated main body 1, and as already mentioned 5 above, the front body 2 has a letter “M” shape and on a front face having anchoring means 2a.

To regulate, control, and adjust the pressure within the user's nostrils, this invention has an adjustment tensioner 3 arranged within the voltage channel 4, extending the adjustment tensioner 3 to one end of the flexible longitudinal portion 1b, 10 having the adjustment tensioner 3 at one front end, an adjustment medium 3a which is anchored to the anchoring means 2a.

Inside the front body 2, there is a motherboard 7 with Bluetooth, microphone, and ultrasonic motor, which via low-power Bluetooth connection, the device has the ability to emit statistics and the current status of the user. The microphone captures sound and mass flow variations of air so that it processes that data, and if necessary, the ultrasonic motor is activated. The Bluetooth connectivity allows external devices such as mobile phones or medical devices to interact with our device.

Through the programming of the motherboard 7 and using communication cables 4 to the modular terminals 5 that can emit magnetic or electrical signals, which stimulate the soft palate of the patient.

The figures may depict exemplary configurations for the invention, which is done to aid in understanding the features and functionality that can be included in the invention. The invention is not restricted to the illustrated architectures or configurations, but can be implemented using a variety of alternative architectures and configurations. Additionally, although the invention is described above in terms of various exemplary embodiments and implementations, it should be understood that the various features and functionality described in one or more of the individual embodiments with which they are described, but instead can be applied, alone or in some combination, to one or more of the other embodiments of the invention, whether or not such embodiments are described and whether or not such features are presented as being a part of a described embodiment. Thus the breadth and scope of the present invention, especially in the following claims, should not be limited by any of the above-described exemplary embodiments.

Terms and phrases used in this document, and variations thereof, unless otherwise expressly stated, should be construed as open ended as opposed to limiting. As examples of the foregoing: the term “including” should be read as mean “including, without limitation” or the like; the term “example” is used to provide exemplary instances of the item in discussion, not an exhaustive or limiting list thereof; and adjectives such as “conventional,” “traditional,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although item, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

I claim:

1. A device for preventing sleep apnea and snoring, comprising:
an elongated main body with a rigid longitudinal portion and a flexible longitudinal portion that is removed from the rigid longitudinal portion, having said elongated main body a tension channel extending over the entire length of it;
a front body disposed at one end of the rigid longitudinal portion of the elongated main body, having the front body on a front face with anchoring means;
an adjustment tensioner arranged within the tension channel, extending the adjustment tensioner to one end of the flexible longitudinal portion, having the adjustment tensioner at one front end, an adjustment medium which is anchored to the anchoring means.

2. The device for preventing sleep apnea and snoring by the 15 claim 1, wherein the frontal body 2 has a substantial form of “M”.

3. The device for preventing sleep apnea and snoring by the claim 1, wherein the anchoring means are more than one support hook secured to the adjustment means.

4. The device for preventing sleep apnea and snoring by the claim 1, which comprises a programmable motherboard so that through communication cables, it activates modular terminals that can emit magnetic or electrical signals, which stimulate the patient's soft palate.

5. The device for preventing sleep apnea and snoring by the claim 1, wherein the elongated main body has a dimension of between 5 cm to 10 cm.
